(19) United States
(12) Patent Application Publication
(10) Pub. No.: US 2003/0220255 A1
(43) Pub. Date: Nov. 27, 2003

(54) GLP-1 agonist and cardiovascular complications

(76) Inventors: Liselotte Bjerre Knudsen, Valby (DK); Bidda Charlotte Rolin, Naerum (DK); Richard David Carr, Vaerlose (DK); Johan Selmer, Farum (DK); Jens Larsen, Fredensborg (DK); Bodil Elbrond, Kobenhavn (DK); Lars Bo Nielsen, Virum (DK); Christina Christoffersen, Kobenhavn (DK)

Correspondence Address: Reza Green, Esq., Novo Nordisk Pharmaceuticals, Inc., 100 College Road West, Princeton, NJ, 08540, US

(21) Appl. No.: 10/406,426

(22) Filed: Apr. 3, 2003

Related U.S. Application Data
(60) Provisional application No. 60/375,255, filed on Apr. 23, 2002.

(30) Foreign Application Priority Data
Apr. 4, 2002 (DK) PA 2002 00499

(51) Int. Cl. A61K 38/26

(52) U.S. Cl. 514/12

(57) ABSTRACT

Methods and uses for the treatment and prevention of cardiac and cardiovascular diseases comprising administration of a GLP-1 agonist.

### CROSS REFERENCE TO RELATED APPLICATIONS

This application claims priority under 35 U.S.C. 119 of Danish application no. PA 2002 00499 filed Apr. 4, 2002, and to U.S. provisional application No. 60/375255 filed Apr. 23, 2002, the contents of which are fully incorporated herein by reference.

### FIELD OF THE INVENTION

The present invention relates to methods for treatment and/or prevention of cardiac and cardiovascular diseases. More specifically, the methods and uses of the invention pertain to administration of a GLP-1 agonist.

### BACKGROUND OF THE INVENTION

Even well controlled diabetics acquire a range of complications and diseases which lowers quality of life and increases morbidity and mortality. Among these complications and diseases are the cardiac and cardiovascular diseases.

The natriuretic peptide family plays a pivotal role in the homeostasis of intravascular fluid balance and in the maintenance of cardiovascular hemodynamics (N. Engl. J. Med. 1998:339:321-328). The natriuretic peptide family includes Atrial Natriuretic Peptide (ANP), Brain Natriuretic Peptide (BNP) and type C natriuretic peptide. Brain natriuretic peptide (BNP) was discovered in 1988 (Nature 1988:332(6159):78-81) in porcine brain tissue. BNP is currently receiving an increasing amount of attention as a marker of cardiac diseases (Scan. J. Clic Invest 2001;61;S234:47-51, Diabetes Care 2001:24(11):2019, Lancet 1998:351(9095):9-13, Lancet 1997:350(9088):1349-1353, Cardivasc Res 2001:51(3): 442-449).

Human GLP-1 is a 37 amino acid residue peptide originating from preproglucagon which is synthesized i.a. in the L-cells in the distal ileum, in the pancreas and in the brain. GLP-1 is an important gut hormone with regulatory function in glucose metabolism and gastrointestinal secretion and metabolism. Processing of preproglucagon to give GLP-1 (7-36)amide, GLP-1 (7-37) and GLP-2 occurs mainly in the L-cells. A simple system is used to describe fragments and analogues of this peptide. Thus, for example, Gly⁸-GLP-1 (7-37) designates a fragment of GLP-1 formally derived from GLP-1 by deleting the amino acid residues Nos: 1 to 6 and substituting the naturally occurring amino acid residue in position 8 (Ala) by Gly. Similarly, Lys³⁴(N^{ε}-tetradecanoyl)-GLP-1(7-37) designates GLP-1(7-37) wherein the ε-amino group of the Lys residue in position 34 has been tetradecanoylated. PCT publications WO 98/08871 and WO 99/43706 disclose stable derivatives of GLP-1 analogues, which have a lipophilic substituent. These stable derivatives of GLP-1 analogues have a protracted profile of action compared to the corresponding GLP-1 analogues. Small non-peptidyl organic molecules are also known to be GLP-1 agonists.

GLP-1 agonists have earlier been described to be useful for treating hyperglycemia (WO 98/08871), for treating dyslipidemia (WO 01/66135), for reducing morbidity and mortality after myocardial infarct (MI) (U.S. Pat. No. 6,277,819), for treating acute coronary syndrome (ACS), unstable angina (UA), non-Q-wave cardiac necrosis (NQCN) and Q-wave MI (QMI) (WO 01/89554), for reducing morbidity and mortality after stroke (WO 00/16797) as well as for increasing urine flow (WO 99/40788).

We have found that GLP-1 agonists are effective in lowering BNP in heart tissue, thus being useful for the treatment and prevention of a range of early cardiac and early cardiovascular diseases.

### SUMMARY OF THE INVENTION

One object of the invention is to provide methods which can effectively be used in the treatment and prevention of early cardiac and early cardiovascular diseases such as left ventricular hypertrophy, coronary artery disease, essential hypertension, acute hypertensive emergency, cardiomyopathy, heart insufficiency, exercise tolerance, chronic heart failure, arrhythmia, cardiac dysrhythmia, syncopy, atheroschlerosis, mild chronic heart failure, angina pectoris, cardiac bypass reocclusion, intermittent claudication (atheroschlerosis oblitterens), diastolic dysfunction and systolic dysfuntion.

A further object of the invention is to provide methods which can effectively be used for reducing the level of BNP in plasma and/or in heart tissue.

One such method comprises the administration of a GLP-1 agonist or a pharmaceutically acceptable salt thereof to a patient in need thereof. Another method comprises the administration of a GLP-1 agonist and one or more additional pharmaceutical agents to a patient.

In one embodiment of the invention, the GLP-1 agonist is a stable derivative of a GLP-1 analogue. In a preferred embodiment the GLP-1 agonist is a GLP-1 analogue with a lipophilic substituent, preferably Arg³⁴, Lys²⁶(N^{ε}-(γ-Glu(N^{α}-hexadecanoyl)))-GLP-1 (7-37).

### DESCRIPTION OF THE INVENTION

GLP-1 agonists have well known effects on blood glucose and plasma lipids. It has been discovered that GLP-1 agonists lowers the concentration of BNP in cardiac tissue. Thus, GLP-1 agonists are potential drugs for the treatment and prevention of a wide range of cardiac and cardiovascular diseases.

Accordingly, one aspect of the present invention is the use of a GLP-1 agonist or a pharmaceutically acceptable salt thereof for the preparation of a pharmaceutical composition for the treatment or prevention of an early cardiac or early cardiovascular disease in a patient in need thereof. By an early cardiac or early cardiovascular disease is meant a stage of disease prior to stroke or myocardial infarct.

In this application “treatment” is defined as the management and care of a patient for the purpose of combating or alleviating the disease, condition, or disorder and includes the administration of the active compound to alleviate the symptoms or complications, or eliminating the disease, condition, or disorder. In this application “prevention” is defined as the management and care of a patient for the purpose of combating the disease, condition, or disorder and includes the administration of the active compounds to prevent the onset of the symptoms or complications.

Within the context of the present invention, “a GLP-1 agonist” is understood to refer to any compound, including petides and non-peptide compounds, which fully or partially activates the human GLP-1 receptor.

In one embodiment the early cardiac or early cardiovascular disease is selected from the group consisting of left ventricular hypertrophy, coronary artery disease, essential hypertension, acute hypertensive emergency, cardiomyopathy, heart insufficiency, exercise tolerance, chronic heart failure, arrhythmia, cardiac dysrhythmia, syncopy, atheroschlerosis, mild chronic heart failure, angina pectoris, cardiac bypass reocclusion, intermittent claudication (atheroschlerosis oblitterens), diastolic dysfunction and systolic dysfuntion.

In another embodiment the early cardiac or early cardiovascular disease is atherosclerosis.

In another embodiment the early cardiac or early cardiovascular disease is left ventricular hypertrophy.

In another embodiment the early cardiac or early cardiovascular disease is coronary artery disease.

In another embodiment the early cardiac or early cardiovascular disease is essential hypertension.

In another embodiment the early cardiac or early cardiovascular disease is acute hypertensive emergency.

In another embodiment the early cardiac or early cardiovascular disease is cardiomyopathy.

In another embodiment the early cardiac or early cardiovascular disease is heart insufficiency.

In another embodiment the early cardiac or early cardiovascular disease is exercise tolerance.

In another embodiment the early cardiac or early cardiovascular disease is chronic heart failure.

In another embodiment the early cardiac or early cardiovascular disease is arrhythmia.

In another embodiment the early cardiac or early cardiovascular disease is cardiac dysrhythmia.

In another embodiment the early cardiac or early cardiovascular disease is syncopy.

In another embodiment the early cardiac or early cardiovascular disease is mild chronic heart failure.

In another embodiment the early cardiac or early cardiovascular disease is angina pectoris

In another embodiment the early cardiac or early cardiovascular disease is cardiac bypass reocclusion.

In another embodiment the early cardiac or early cardiovascular disease is intermittent claudication (atheroschlerosis oblitterens).

In another embodiment the early cardiac or early cardiovascular disease is diastolic dysfunction.

In another embodiment the early cardiac or early cardiovascular disease is systolic dysfunction.

In a second aspect the present invention relates to the use of a GLP-1 agonist or a pharmaceutically acceptable salt thereof for the preparation of a pharmaceutical composition for reducing the level of BNP in plasma and/or in heart tissue of a patient in need thereof.

In one embodiment the patient suffers from a disease selected from the group consisting of left ventricular hypertrophy, coronary artery disease, essential hypertension, acute hypertensive emergency, cardiomyopathy, heart insufficiency, exercise tolerance, chronic heart failure, arrhythmia, cardiac dysrhythmia, syncopy, atheroschlerosis, mild chronic heart failure, angina pectoris, cardiac bypass reocclusion, intermittent claudication (atheroschlerosis oblitterens), diastolic dysfunction and systolic dysfuntion.

In another embodiment the patient suffers from a disease selected from the group consisting of myocardial infarct, acute coronary syndrome, unstable angina, non-Q-wave cardiac necrosis, Q-wave myocardial infarct and morbidity after stroke.

In another embodiment of the use according to the first and second aspect of the invention, the patient is a diabetic patient.

In yet another embodiment the use according to the first and second aspect of the invention, the patient is a non-diabetic patient.

In yet another embodiment the GLP-1 agonist is selected from the group consisting of GLP-1(7-36)-amide, GLP-1(7-37), a GLP-1(7-36)-amide analogue, a GLP-1(7-37) analogue, or a derivative of any of these.

In yet another embodiment the GLP-1 agonist is a derivative of GLP-1(7-36)-amide, GLP-1(7-37), a GLP-1(7-36)-amide analogue or a GLP-1(7-37) analogue, which comprises a lipophilic substituent.

In yet another embodiment the GLP-1 agonist is Arg³⁴, Lys²⁶(N^{ε}-(γ-Glu(N^{α}-hexadecanoyl)))-GLP-1(7-37).

In yet another embodiment the GLP-1 agonist is selected from the group consisting of Gly⁸-GLP-1(7-36)-amide, Gly⁸-GLP-1(7-37), Val⁸-GLP-1(7-36)-amide, Val⁸-GLP-1(7-37), Val⁸Asp²²-GLP-1(7-36)-amide, Val⁸Asp²²-GLP-1(7-37), Val⁸Glu²²-GLP-1(7-36)-amide, Val⁸Glu²²-GLP-1(7-37), Val⁸Lys²²-GLP-1(7-36)-amide, Val⁸Lys²²-GLP-1(7-37), Val⁸Arg²²-GLP-1(7-36)-amide, Val⁸Arg²²-GLP-1(7-37), Val⁸His²²-GLP-1(7-36)-amide, Val⁸His²²-GLP-1(7-37), analogues thereof and derivatives of any of these.

In yet another embodiment the GLP-1 agonist is selected from the group consisting of Arg²⁶-GLP-1(7-37); Arg³⁴-GLP-1(7-37); Lys³⁶-GLP-1(7-37); Arg^{26,34}Lys³⁶-GLP-1(7-37); Arg^{26,34}-GLP-1(7-37); Arg^{26,34}Lys⁴⁰-GLP-1(7-37); Arg²⁶Lys³⁶-GLP-1(7-37); Arg³⁴Lys³⁴-GLP-1(7-37); Val⁸Arg²²-GLP-1(7-37); Met⁸Arg²²-GLP-1(7-37); Gly⁸His²²-GLP-1(7-37); Val⁸His²²-GLP-1(7-37); Met⁸His²²-GLP-1(7-37); His³⁷-GLP-1(7-37); Gly⁸-GLP-1(7-37); Val⁸-GLP-1(7-37); Met⁸-GLP-1(7-37); Gly⁸Asp²²-GLP-1(7-37); Val⁸Asp²²-GLP-1(7-37); Met⁸Asp²²-GLP-1(7-37); Gly⁸Glu²²-GLP-1(7-37); Val⁸Glu²²-GLP-1(7-37); Met⁸Glu²²-GLP-1(7-37); Gly⁸Lys²²-GLP-1(7-37); Val⁸Lys²²-GLP-1(7-37); Met⁸Lys²²-GLP-1(7-37); Gly⁸Arg²²-GLP-1(7-37); Val⁸Lys²²His³⁷-GLP-1(7-37); Gly⁸Glu²²His³⁷-GLP-1(7-37); Val⁸Glu²²His³⁷-GLP-1(7-37); Met⁸Glu²²His³⁷-GLP-1(7-37); Gly⁸Lys²²His³⁷-GLP-1(7-37); Met⁸Lys²²His³⁷-GLP-1(7-37); Gly⁸Arg²²His³⁷-GLP-1(7-37); Val⁸Arg²²His³⁷-GLP-1(7-37); Met⁸Arg²²His³⁷-GLP-1(7-37); Gly⁸His²²His³⁷-GLP-1(7-37); Val⁸His²²His³⁷-GLP-1(7-37); Met⁸His²²His³⁷-GLP-1(7-37); Gly⁸His³⁷-GLP-1(7-37); Val⁸His³⁷-GLP-1(7-37); Met⁸His³⁷-GLP-1(7-37); Gly⁸Asp²² His³⁷-GLP-1(7-37); Val⁸Asp²²His³⁷-GLP-1(7-37); Met⁸Asp²²His³⁷-GLP-1(7-37); Arg²⁶-GLP-1(7-36)-amide; Arg³⁴-GLP-1(7-36)-amide; Lys³⁶-GLP-1(7-36)-amide; Arg^{26,34}Lys³⁶-GLP-1(7-36)-amide; Arg^{26,34}GLP-1(7-36)-amide; Arg^{26,34}Lys⁴⁰-GLP-1(7-36)-amide; Arg²⁶Lys³⁶-GLP-1(7-36)-amide; Arg³⁴Lys³⁶-GLP-1(7-36)-amide; Gly⁸-GLP-1(7-36)-amide; Val⁸-GLP-1(7-36)-amide; Met⁸-GLP-1(7-36)-amide; Gly⁸Asp²²-GLP-1(7-36)-amide; Gly⁸Glu²²His³⁷-GLP-1(7-36)-amide; Val⁸Asp²²GLP-1(7-36)-amide; Met⁸Asp²²-GLP-1(7-36)-amide; Gly⁸Glu²²-GLP-1(7-36)-amide; Val⁸Glu²²-GLP-1(7-36)-amide; Met⁸Glu²²-GLP-1(7-36)-amide; Gly⁸Lys²²-GLP-1(7-36)-amide; Val⁸Lys²²-GLP-1(7-36)-amide; Met⁸Lys²²-GLP-1(7-36)-amide; Gly⁸His²²His³⁷-GLP-1(7-36)-amide; Gly⁸Arg²²-GLP-1(7-36)-amide; Val⁸Arg²²-GLP-1(7-36)-amide; Met⁸Arg²²-GLP-1(7-36)-amide; Gly⁸His²²-GLP-1(7-36)-amide; Val⁸His²²-GLP-1(7-36)-amide; Met⁸His²²-GLP-1(7-36)-amide; His³⁷-GLP-1(7-36)-amide; Val⁸Arg²²His³⁷-GLP-1(7-36)-amide; Met⁸Arg²²His³⁷-GLP-1(7-36)-amide; Gly⁸His³⁷-GLP-1(7-36)-amide; Val⁸His³⁷-GLP-1(7-36)-amide; Met⁸His³⁷-GLP-1(7-36)-amide; Gly⁸Asp²²His³⁷-GLP-1(7-36)-amide; Val⁸Asp²²His³⁷-GLP-1(7-36)-amide; Met⁸Asp²²His³⁷-GLP-1(7-36)-amide; Val⁸Glu²²His³⁷-GLP-1(7-36)-amide; Met⁸Glu²²His³⁷-GLP-1(7-36)-amide; Gly⁸Lys²²His³⁷-GLP-1(7-36)-amide; Val⁸Lys²²His³⁷-GLP-1(7-36)-amide; Met⁸Lys²²His³⁷-GLP-1(7-36)-amide; Gly⁸Arg²²His³⁷-GLP-1(7-36)-amide; Val⁸His²²His³⁷-GLP-1(7-36)-amide; Met⁸His²²His³⁷-GLP-1(7-36)-amide; and derivatives thereof.

In yet another embodiment the GLP-1 agonist is selected from the group consisting of Val⁸Trp¹⁹Glu²²-GLP-1(7-37), Val⁸Glu²²Val²⁵-GLP-1(7-37), Val⁸Tyr¹⁶Glu²²-GLP-1(7-37), Val⁸Trp¹⁶Glu²²-GLP-1(7-37), Val⁸Leu¹⁶Glu²²-GLP-1(7-37), Val⁸Tyr¹⁸Glu²²-GLP-1(7-37), Val⁸Glu²²His³⁷-GLP-1(7-37), Val⁸Glu²²IIe³³-GLP-1(7-37), Val⁸Trp¹⁶Glu²²Val²⁵IIe³³-GLP-1(7-37), Val⁸Trp¹⁶Glu²²IIe³³-GLP-1(7-37), Val⁸GIu²²Val²⁵IIe³³-GLP-1(7-37), Val⁸Trp¹⁶Glu²²Val²⁵-GLP-1(7-37), analogues thereof and derivatives of any of these.

In yet another embodiment the GLP-1 agonist is a stable GLP-1 analogue/derivative. Throughout this application a “stable GLP-1 analogue/derivative” means a GLP-1 analogue or a derivative of a GLP-1 analogue which exhibits an in vivo plasma elimination half-life of at least 10 hours in man, as determined by the method described below. Examples of stable GLP-1 analogue/derivatives can be found in WO 98/08871 and WO 99/43706. The method for determination of plasma elimination half-life of a compound in man is: The compound is dissolved in an isotonic buffer, pH 7.4, PBS or any other suitable buffer. The dose is injected peripherally, preferably in the abdominal or upper thigh. Blood samples for determination of active compound are taken at frequent intervals, and for a sufficient duration to cover the terminal elimination part (e.g. Pre-dose, 1, 2, 3, 4, 5, 6, 7, 8, 10, 12, 24 (day 2), 36 (day 2), 48 (day 3), 60 (day 3), 72 (day 4) and 84 (day 4) hours post dose). Determination of the concentration of active compound is performed as described in Wilken et al., Diabetologia 43(51):A143, 2000. Derived pharmacokinetic parameteres are calculated from the concentration-time data for each individual subject by use of non-compartmental methods, using the commercially available software WinNonlin Version 2.1 (Pharsight, Cary, N.C., USA). The terminal elimination rate constant is estimated by log-linear regression on the terminal log-linear part of the concentration-time curve, and used for calculating the elimination half-life.

Stable GLP-1 analogues and derivatives are disclosed in WO 98/08871 (analogues with lipophilic substituent) and in WO 02/46227 (analogues fused to serum albumin or to Fc portion of an Ig).

In yet another embodiment the GLP-1 agonist is exendin-4, an exendin-4 analogue or a derivative of any of these.

In yet another embodiment the GLP-1 agonist is a stable exendin-4 analogue/derivative. The term “stable exendin-4 analogue/derivative”, as used herein refers to an exendin-4(1-39) analogue or a derivative of an exendin-4(1-39) analogue which exhibits an in vivo plasma elimination half-life of at least 10 hours in man, as determined by the method described above.

Examples of exendins as well as analogues, derivatives, and fragments thereof to be included within the present invention are those disclosed in WO 97/46584, U.S. Pat. No. 5,424,286 and WO 01/04156. U.S. Pat. No. 5,424,286 describes a method for stimulating insulin release with an exendin polypeptide. The exendin polypeptides disclosed include HGEGTFTSDLSKQMEEEAVRLFIEWLKNGGX; wherein X=P or Y, and HX1X2GTFITSDLSKQMEEEAVRLFIEWLKNGGPSSGAPPPS; wherein X1X2=SD (exendin-3) or GE (exendin-4)). WO 97/46584 describes truncated versions of exendin peptide(s). The disclosed peptides increase secretion and biosynthesis of insulin, but reduce those of glucagon. WO 01/04156 describes exendin-4 analogues and derivatives as well as the preparation of these molecules. Exendin-4 analogues stabilized by fusion to serum albumin or Fc portion of an Ig are disclosed in WO 02/46227.

In still another embodiment the GLP-1 agonist is selected from the GLP-1 agonists disclosed in WO 00/42026.

The present invention also encompasses pharmaceutically acceptable salts of the GLP-1 agonists. Such salts include pharmaceutically acceptable acid addition salts, pharmaceutically acceptable metal salts, ammonium and alkylated ammonium salts. Acid addition salts include salts of inorganic acids as well as organic acids. Representative examples of suitable inorganic acids include hydrochloric, hydrobromic, hydroiodic, phosphoric, sulfuric, nitric acids and the like. Representative examples of suitable organic acids include formic, acetic, trichloroacetic, trifluoroacetic, propionic, benzoic, cinnamic, citric, fumaric, glycolic, lactic, maleic, malic, malonic, mandelic, oxalic, picric, pyruvic, salicylic, succinic, methanesulfonic, ethanesulfonic, tartaric, ascorbic, pamoic, bismethylene salicylic, ethanedisulfonic, gluconic, citraconic, aspartic, stearic, palmitic, EDTA, glycolic, p-aminobenzoic, glutamic, benzenesulfonic, p-toluenesulfonic acids and the like. Further examples of pharmaceutically acceptable inorganic or organic acid addition salts include the pharmaceutically acceptable salts listed in J. Pharm. Sci. 1977, 66, 2. Examples of metal salts include lithium, sodium, potassium, magnesium salts and the like. Examples of ammonium and alkylated ammonium salts include ammonium, methylammonium, dimethylammonium, trimethylammonium, ethylammonium, hydroxyethylammonium, diethylammonium, butylammonium, tetramethylammonium salts and the like.

Also intended as pharmaceutically acceptable acid addition salts are the hydrates which the present GLP-1 agonists are able to form.

Peptide GLP-1 compounds can be produced by appropriate derivatization of an appropriate peptide backbone which has been produced by recombinant DNA technology or by peptide synthesis (e.g. Merrifield-type solid phase synthesis) as known in the art of peptide synthesis and peptide chemistry.

The route of administration may be any route, which effectively transports the active compound to the appropriate or desired -site of action, such as oral, nasal, buccal, pulmonal, transdermal or parenteral.

Pharmaceutical compositions (or medicaments) containing a GLP-1 agonist, such as Arg³⁴, Lys²⁶ (N^{ε}-(γ-Glu(N^{α}-hexadecanoyl)))-GLP-1(7-37), may be administered parenterally to patients in need of such a treatment. Parenteral administration may be performed by subcutaneous, intramuscular or intravenous injection by means of a syringe, optionally a pen-like syringe. Alternatively, parenteral administration can be performed by means of an infusion pump. A further option is a composition which may be a powder or a liquid for the administration of GLP-1 agonists in the form of a nasal or pulmonal spray. As a still further option, the GLP-1 agonist can also be administered transdermally, e.g. from a patch, optionally a iontophoretic patch, or transmucosally, e.g. bucally. The above-mentioned possible ways to administer GLP-1 agonists are not considered as limiting the scope of the invention.

Pharmaceutical compositions containing GLP-1 agonists, such as Arg³⁴, Lys²⁶(N^{ε}-(γ-Glu(N^{α}-hexadecanoyl)))-GLP-1(7-37), may be prepared by conventional techniques, e.g. as described in Remington's Pharmaceutical Sciences, 1985 or in Remington: The Science and Practice of Pharmacy, 19^{th} edition, 1995.

Thus, the injectable compositions of GLP-1 agonists can be prepared using the conventional techniques of the pharmaceutical industry which involves dissolving and mixing the ingredients as appropriate to give the desired end product.

According to one procedure, e.g. Arg³⁴, Lys²⁶(N^{ε}-(γ-Glu(N^{α}-hexadecanoyl)))-GLP-1(7-37) is dissolved in an amount of water which is somewhat less than the final volume of the composition to be prepared. An isotonic agent, a preservative and a buffer are added as required and the pH value of the solution is adjusted—if necessary—using an acid, e.g. hydrochloric acid, or a base, e.g. aqueous sodium hydroxide as needed. Finally, the volume of the solution is adjusted with water to give the desired concentration of the ingredients.

Examples of isotonic agents are sodium chloride, mannitol and glycerol.

Examples of preservatives are phenol, m-cresol, methyl p-hydroxybenzoate and benzyl alcohol.

Examples of suitable buffers are sodium acetate and sodium phosphate.

Further to the above-mentioned components, solutions containing a peptide GLP-1 agonist may also contain a surfactant in order to improve the solubility and/or the stability of the peptide.

According to one embodiment of the present invention, the GLP-1 agonist is provided in the form of a composition suitable for administration by injection. Such a composition can either be an injectable solution ready for use or it can be an amount of a solid composition, e.g. a lyophilised product, which has to be dissolved in a solvent before it can be injected. The injectable solution preferably contains not less than about 0.1 mg/ml, typically from 0.1 mg/ml to 5 mg/ml, such as from 1 mg/ml to 5 mg/ml of stable derivative of GLP-1 analogue.

Derivatives of GLP-1 analogues such as Arg³⁴, Lys²⁶(N^{ε}-(γ-Glu(N^{α}-hexadecanoyl)))GLP-1(7-37) can be used in the treatment of various cardiac and cardiovascular diseases. The optimal dose level for any patient (effective amount) will depend on the disease to be treated and on a variety of factors including the efficacy of the specific GLP-1 agonist employed, the age, body weight, physical activity, and diet of the patient, on a possible combination with other drugs, and on the severity of the case.

In yet another embodiment the pharmaceutical composition is a parenteral composition.

In yet another embodiment the pharmaceutical composition comprises a buffer, an isotonicity agent and a preservative.

In yet another embodiment the pharmaceutical composition is to be administered intravenously or subcutaneously.

In yet another embodiment the pharmaceutical composition is administered by injection.

In yet another embodiment the pharmaceutical composition is administered by infusion.

In yet another embodiment the dosage of GLP-1 agonist is from about 0.5 μg/kg/day to about 20 μg/kg/day.

In yet another embodiment the dosage of GLP-1 agonist is from about 0.1 μg/kg/day to about 2 μg/kg/day.

The pharmaceutical composition may be administered continuously by infusion, one or more times daily such as one to three times daily, or at longer intervals such as weekly or monthly in the form of a depot preparation.

In yet another embodiment the pharmaceutical composition is administered to the patient for more than 1 week, preferably for more than 4 weeks, more preferred for more than 3 months, and even more preferred for more than 6 months.

In another aspect the present invention relates to the use of a GLP-1 agonist, wherein one or more further pharmaceutical agents are administered to the patient. These further pharmaceutical agents may be administered simultaneously, separately or sequentially with the GLP-1 agonist.

In one embodiment said further pharmaceutical agent is selected from the group consisting of anti-diabetic agents, anti-obesity agents, lipid modulating agents, anti-hypertensive agents and antiosteoporosis agents.

In a further aspect of the invention treatment of a patient with the present compounds is combined with diet and/or exercise.

Relevant antidiabetic agents include insulin, insulin analogues and derivatives such as those disclosed in EP 0 792 290 (Novo Nordisk A/S), eg N^{εB29}-tetradecanoyl des (B30) human insulin, EP 0 214 826 and EP 0 705 275 (Novo Nordisk A/S), eg Asp^{B28} human insulin, U.S. Pat. No. 5,504,188 (Eli Lilly), eg Lys^{B28} Pro^{B29} human insulin, EP 0 368 187 (Aventis), eg Lantus®.

The orally active hypoglycaemic agents preferably comprise imidazolines, sulfonylureas, biguanides, meglitinides, oxadiazolidinediones, thiazolidinediones, insulin sensitizers, α-glucosidase inhibitors, agents acting on the ATP-dependent potassium channel of the β-cells eg potassium channel openers such as those disclosed in WO 97/26265, WO 99/03861 and WO 00/37474 (Novo Nordisk A/S), or mitiglinide, or a potassium channel blocker, such as BTS-67582, nateglinide, glucagon antagonists such as those disclosed in WO 99/01423 and WO 00/39088 (Novo Nordisk A/S and Agouron Pharmaceuticals, Inc.), GLP-1 agonists such as those disclosed in WO 00/42026 (Novo Nordisk A/S and Agouron Pharmaceuticals, Inc.), DPP-IV (dipeptidyl peptidase-IV) inhibitors, PTPase (protein tyrosine phosphatase) inhibitors, inhibitors of hepatic enzymes involved in stimulation of gluconeogenesis and/or glycogenolysis, glucose uptake modulators, GSK-3 (glycogen synthase kinase-3) inhibitors, compounds modifying the lipid metabolism such as antilipidemic agents, compounds lowering food intake, PPAR (peroxisome proliferator-activated receptor) and RXR (retinoid X receptor) agonists, such as ALRT-268, LG-1268 or LG-1069.

Relevant anti-obesity agents include CART (cocaine amphetamine regulated transcript) agonists, NPY (neuropeptide Y) antagonists, MC4 (melanocortin 4) agonists, MC3 (melanocortin 3) agonists, orexin antagonists, TNF (tumor necrosis factor) agonists, CRF (corticotropin releasing factor) agonists, CRF BP (corticotropin releasing factor binding protein) antagonists, urocortin agonists, β3 adrenergic agonists such as CL-316243, AJ-9677, GW-0604, LY362884, LY377267 or AZ-40140, MSH (melanocyte-stimulating hormone) agonists, MCH (melanocyte-concentrating hormone) antagonists, CCK (cholecystokinin) agonists, serotonin re-uptake inhibitors such as fluoxetine, seroxat or citalopram, serotonin and noradrenaline re-uptake inhibitors, mixed serotonin and noradrenergic compounds, 5HT (serotonin) agonists, bombesin agonists, galanin antagonists, growth hormone, growth factors such as prolactin or placental lactogen, growth hormone releasing compounds, TRH (thyreotropin releasing hormone) agonists, UCP 2 or 3 (uncoupling protein 2 or 3) modulators, leptin agonists, DA agonists (bromocriptin, doprexin), lipase/amylase inhibitors, PPAR (peroxisome proliferator-activated receptor) modulators, RXR (retinoid X receptor) modulators, TRβ agonists, AGRP (Agouti related protein) inhibitors, opioid antagonists (such as naltrexone), H3 histamine antagonists and ciliary neurotrophic factor.

Relevant lipid modulating agents include cholestyramine, colestipol, clofibrate, gemfibrozil, lovastatin, pravastatin, simvastatin, probucol and dextrothyroxine.

In one embodiment the anti-hypertensive agent is an angiotensin converting enzyme inhibitor.

In yet another embodiment the angiotensin converting enzyme inhibitor is selected from the group consisting of captopril, enalapril, fosinoprol, lisnoprol, quinapril, ramipril and spirapril.

In yet another embodiment the anti-hypertensive agent is an angiotensin II receptor antagonist, e.g. losartan.

In yet another embodiment the anti-hypertensive agent is a non-subtype-selective β-adrenergic antagonist.

In yet another embodiment the non-subtype-selective β-adrenergic antagonist is selected from the group consisting of propranolol, nadolol, timolol and pindolol.

In yet another embodiment the antihypertensive agent is a selective β₁-adrenergic antagonist.

In yet another embodiment the selective β₁-adrenergic antagonist is selected from the group consisting of metoprolol, atenolol, esmolol and acebutolol.

In another aspect the present invention relates to a method for the treatment or prevention of an early cardiac or early cardiocascular disease, which method comprises administration of an effective amount of a GLP-1 agonist or a pharmaceutically acceptable salt thereof to a patient in need thereof.

In one embodiment of the method the early cardiac or early cardiovascular disease is selected from the group consisting of left ventricular hypertrophy, coronary artery disease, essential hypertension, acute hypertensive emergency, cardiomyopathy, heart insufficiency, exercise tolerance, chronic heart failure, arrhythmia, cardiac dysrhythmia, syncopy, atheroschlerosis, mild chronic heart failure, angina pectoris, cardiac bypass reocclusion, intermittent claudication (atheroschlerosis oblitterens), diastolic dysfunction and systolic dysfunction.

In another aspect the invention relates to a method for reducing the level of BNP in plasma and/or in heart tissue, which method comprises administration of an effective amount of a GLP-1 agonist or a pharmaceutically acceptable salt thereof to a patient in need thereof.

In one embodiment the patient suffers from a disease selected from the group consisting of left ventricular hypertrophy, coronary artery disease, essential hypertension, acute hypertensive emergency, cardiomyopathy, heart insufficiency, exercise tolerance, chronic heart failure, arrhythmia, cardiac dysrhythmia, syncopy, atheroschlerosis, mild chronic heart failure, angina pectoris, cardiac bypass reocclusion, intermittent claudication (atheroschlerosis oblitterens), diastolic dysfunction and systolic dysfuntion.

In another embodiment the patient suffers from a disease selected from the group consisting of myocardial infarct, acute coronary syndrome, unstable angina, non-Q-wave cardiac necrosis, Q-wave myocardial infarct and morbidity after stroke.

In another embodiment of the methods the patient is a diabetic patient.

In yet another embodiment of the methods the patient is a non-diabetic patient.

In yet another embodiment the GLP-1 agonist is selected from the group consisting of GLP-1(7-36)-amide, GLP-1(7-37), a GLP-1(7-36)-amide analogue, a GLP-1(7-37) analogue, or a derivative of any of these.

In yet another embodiment the GLP-1 agonist is a derivative of GLP-1(7-36)-amide, GLP-1(7-37), a GLP-1(7-36)-amide analogue or a GLP-1(7-37) analogue, which comprises a lipophilic substituent.

In yet another embodiment the GLP-1 agonist is Arg³⁴, Lys²⁶(N^{ε}-(γ-Glu(N^{α}-hexadecanoyl)))-GLP-1(7-37).

In yet another embodiment the GLP-1 agonist is selected from the group consisting of Gly⁸-GLP-1(7-36)-amide, Gly⁸-GLP-1(7-37), Val⁸-GLP-1(7-36)-amide, Val⁸-GLP-1(7-37), Val⁸Asp²²-GLP-1(7-36)-amide, Val⁸Asp²²-GLP-1(7-37), Val⁸Glu²²-GLP-1(7-36)-amide, Val⁸Glu²²-GLP-1(7-37), Val⁸Lys²²-GLP-1(7-36)-amide, Val⁸Lys²²-GLP-1(7-37), Val⁸Arg²²GLP-1(7-36)-amide, Val⁸Arg²²-GLP-1(7-37), Val⁸His²²-GLP-1(7-36)-amide, Val⁸His²²-GLP-1(7-37), analogues thereof and derivatives of any of these.

In yet another embodiment the GLP-1 agonist is a stable GLP-1 analogue/derivative.

In yet another embodiment the GLP-1 agonist is exendin-4, an exendin-4 analogue or a derivative of any of these.

In yet another embodiment the GLP-1 agonist is a stable exendin-4 analogue/derivative.

In yet another embodiment the pharmaceutical composition is a parenteral composition.

In yet another embodiment the pharmaceutical composition comprises a buffer, an isotonicity agent and a preservative.

In yet another embodiment the pharmaceutical composition is to be administered intravenously or subcutaneously.

In yet another embodiment the pharmaceutical composition is administered by injection.

In yet another embodiment the pharmaceutical composition is administered by infusion.

In yet another embodiment the dosage of GLP-1 agonist is from about 0.5 μg/kg/day to about 20 μg/kg/day.

In yet another embodiment the dosage of GLP-1 agonist is from about 0.1 μg/kg/day to about 2 μg/kg/day.

In yet another embodiment the pharmaceutical composition is administered to the patient for more than 1 week, preferably for more than 4 weeks, more preferred for more than 3 months, and even more preferred for more than 6 months.

In another aspect the invention relates to a method wherein one or more further pharmaceutical agents are administered to the patient.

In one embodiment the further pharmaceutical agent is selected from the group consisting of anti-diabetic agents, anti-obesity agents, lipid modulating agents, anti-hypertensive agents and antiosteoporosis agents.

In another embodiment the anti-hypertensive agent is an angiotensin converting enzyme inhibitor.

In yet another embodiment the angiotensin converting enzyme inhibitor is selected from the group consisting of captopril, enalapril, fosinoprol, lisnoprol, quinapril, ramipril and spirapril.

In yet another embodiment the anti-hypertensive agent is an angiotensin II receptor antagonist, e.g. losartan.

In yet another embodiment the anti-hypertensive agent is a non-subtype-selective β-adrenergic antagonist.

In yet another embodiment the non-subtype-selective β-adrenergic antagonist is selected from the group consisting of propranolol, nadolol, timolol and pindolol.

In yet another embodiment the the antihypertensive agent is a selective β₁-adrenergic antagonist.

In yet another embodiment the selective β₁-adrenergic antagonist is selected from the group consisting of metoprolol, atenolol, esmolol and acebutolol.

### EXAMPLES

### Example 1

Hearts from 12 streptozotocin (STZ)-treated pigs were collected. The pigs were treated with STZ 2 weeks prior to dosing with either the GLP-1 derivative Arg³⁴, Lys²⁶(N^{ε}-(γ-Glu(N^{α}-hexadecanoyl)))-GLP-1(7-37) (NN2211) for 4 weeks, dose 3.3 μg/kg s.c. once daily, or vehicle. STZ-treated pigs were either hyperglycemic or glucose intolerant and had impaired insulin secretion upon oral glucose tolerance tests. BNP mRNA and protein levels in cardiac biopsies were measured with real-time PCR and RIA assays, respectively. BNP mRNA levels were normalized by β-actin mRNA levels.

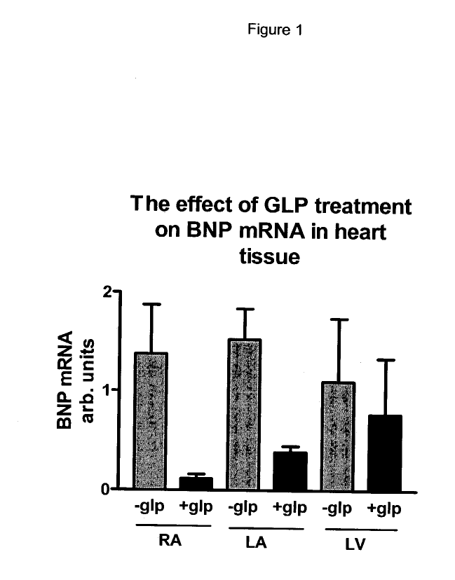

BNP mRNA levels were similar in right atrial (RA), left atrial (LA) and in left ventricular (LV) biopsies from vehicle treated diabetic pigs (−GLP). However, in hearts from NN2211 (+GLP) treated pigs the levels of BNP were significantly lower than in vehicle treated pigs (see FIG. 1).

1. Use of a GLP-1 agonist or a pharmaceutically acceptable salt thereof for the preparation of a pharmaceutical composition for the treatment or prevention of an early cardiac or early cardiovascular disease in a patient in need thereof.

2. The use according to claim 1, wherein said early cardiac or early cardiovascular disease is selected from the group consisting of left ventricular hypertrophy, coronary artery disease, essential hypertension, acute hypertensive emergency, cardiomyopathy, heart insufficiency, exercise tolerance, chronic heart failure, arrhythmia, cardiac dysrhythmia, syncopy, atheroschlerosis, mild chronic heart failure, angina pectoris, cardiac bypass reocclusion, intermittent claudication (atheroschlerosis oblilterens), diastolic dysfunction and systolic dysfunction.

3. Use of a GLP-1 agonist or a pharmaceutically acceptable salt thereof for the preparation of a pharmaceutical composition for reducing the level of BNP in plasma and/or in heart tissue of a patient in need thereof.

4. The use according to any one of claims 1-3, wherein the patient is a diabetic patient.

5. The use according to any one of claims 1-3, wherein the patient is a non-diabetic patient.

6. The use according to any one of claims 1-5, wherein said GLP-1 agonist is selected from the group consisting of GLP-1(7-36)-amide, GLP-1(7-37), a GLP-1(7-36)-amide analogue, a GLP-1(7-37) analogue, or a derivative of any of these.

7. The use according to any one of claims 1-6, wherein the GLP-1 agonist is a derivative of GLP-1(7-36)-amide, GLP-1(7-37), a GLP-1(7-36)-amide analogue or a GLP-1(7-37)analogue, which comprises a lipophilic substituent.

8. The use according to any one of claims 1-7, wherein the GLP-1 agonist is Arg³⁴, Lys²⁶(N^{ε}-(γ-Glu(N^{α}-hexadecanoyl)))-GLP-1(7-37).

9. The use according to claim 6, wherein the GLP-1 agonist is selected from the group consisting of Gly⁸-GLP-1(7-36)-amide, Gly⁸-GLP-1(7-37), Val⁸-GLP-1(7-36)-amide, Val⁸-GLP-1(7-37), Val⁸Asp²²-GLP-1(7-36)-amide, Val⁸Asp²²-GLP-1(7-37), Val⁸Glu²²-GLP-1(7-36)-amide, Val⁸Glu²²-GLP-1(7-37), Val⁸Lys²²-GLP-1(7-36)-amide, Val⁸Lys²²-GLP-1(7-37), Val⁸Arg²²-GLP-1(7-36)-amide, Val⁸Arg²²-GLP-1(7-37), Val⁸His²²-GLP-1(7-36)-amide, Val⁸His²²-GLP-1(7-37), analogues thereof and derivatives of any of these.

10. The use according to any one of claims 6-9, wherein the GLP-1 agonist is a stable GLP-1 analogue/derivative.

11. The use according to any one of claims 1-5, wherein the GLP-1 agonist is exendin-4, an exendin-4 analogue or a derivative of any of these.

12. The use according to claim 11, wherein the GLP-1 agonist is a stable exendin-4 analogue/derivative.

13. The use according to any one of claims 1-12, wherein the pharmaceutical composition is suitable for parenteral administration.

14. The use according to any one of claims 1-13, wherein the pharmaceutical composition comprises a buffer, an isotonicity agent and a preservative.

15. The use according to any one of claims 1-14, wherein the pharmaceutical composition is suitable for intravenous or subcutaneous administration.

16. The use according to any one of claims 13-15, wherein the pharmaceutical composition is suitable for administration by injection.

17. The use according to any one of claims 13-15, wherein the pharmaceutical composition is suitable for administration by infusion.

18. The use according to any one of claims 1-17, wherein the pharmaceutical composition is in a form suitable for delivering a dosage of GLP-1 agonist of from about 0.5 μg/kg/day to about 20 μg/kg/day.

19. The use according to any one of claims 1-17, wherein the pharmaceutical composition is in a form suitable for delivering a dosage of GLP-1 agonist of from about 0.1 μg/kg/day to about 2 μg/kg/day.

20. The use according to any one of claims 1-19, wherein the treatment or prevention is for more than 1 week, preferably for more than 4 weeks, more preferred for more than 3 months, and even more preferred for more than 6 months.

21. The use according to any one of claims 1-20, wherein the treatment or prevention is in combination with one or more further pharmaceutical agents.

22. The use according to claim 21, wherein said further pharmaceutical agent is selected from the group consisting of anti-diabetic agents, anti-obesity agents, lipid modulating agents, anti-hypertensive agents and antiosteoporosis agents.

23. The use according to claim 22, wherein the anti-hypertensive agent is an angiotensin converting enzyme inhibitor.

24. The use according to claim 23, wherein the angiotensin converting enzyme inhibitor is selected from the group consisting of captopril, enalapril, fosinoprol, lisnoprol, quinapril, ramipril and spirapril.

25. The use according to claim 22, wherein the anti-hypertensive agent is an angiotensin II receptor antagonist, e.g. losartan.

26. The use according to claim 22, wherein the anti-hypertensive agent is a non-subtype-selective β-adrenergic antagonist.

27. The use according to claim 26, wherein the non-subtype-selective β-adrenergic antagonist is selected from the group consisting of propranolol, nadolol, timolol and pindolol.

28. The use according to claim 22, wherein the antihypertensive agent is a selective β₁-adrenergic antagonist.

29. The use according to claim 28, wherein the selective β₁-adrenergic antagonist is selected from the group consisting of metoprolol, atenolol, esmolol and acebutolol.

30. A method for treating an early cardiac or early cardiocascular disease, said method comprising administering to a patient in need thereof an effective amount of a GLP-1 agonist or a pharmaceutically acceptable salt thereof.

31. The method according to claim 30, wherein said early cardiac or early cardiovascular disease is selected from the group consisting of left ventricular hypertrophy, coronary artery disease, essential hypertension, acute hypertensive emergency, cardiomyopathy, heart insufficiency, exercise tolerance, chronic heart failure, arrhythmia, cardiac dysrhythmia, syncopy, atheroschlerosis, mild chronic heart failure, angina pectoris, cardiac bypass reocclusion, intermittent claudication (atheroschlerosis oblitterens), diastolic dysfunction and systolic dysfunction

32. A method for reducing the level of brain natriuretic peptide (BNP) in plasma and/or in heart tissue, said method comprising administering to a patient in need thereof an effective amount of a GLP-1 agonist or a pharmaceutically acceptable salt thereof.

33. The method according to claim 30, wherein the patient is a diabetic patient.

34. The method according to claim 30, wherein the patient is a non-diabetic patient.

35. The method according to claim 30, wherein said GLP-1 agonist is selected from the group consisting of GLP-1(7-36)-amide, GLP-1(7-37), a GLP-1(7-36)-amide analogue, a GLP-1(7-37) analogue, or a derivative of any of these.

36. The method according to claim 35, wherein the GLP-1 agonist is a derivative of GLP-1(7-36)-amide, GLP-1(7-37), a GLP-1(7-36)-amide analogue or a GLP-1(7-37) analogue, which comprises a lipophilic substituent.

37. The method according to claim 36, wherein the GLP-1 agonist is Arg³⁴, Lys²⁶(N^{ε}-(γy-Glu(N^{α}-hexadecanoyl)))-GLP-1(7-37).

38. The method according to claim 35, wherein the GLP-1 agonist is selected from the group consisting of Gly⁸-GLP-1(7-36)-amide, Gly⁸-GLP-1(7-37), Val⁸-GLP-1(7-36)-amide, Val⁸GLP-1(7-37), Val⁸Asp²²-GLP-1(7-36)-amide, Val⁸Asp²²-GLP-1(7-37), Val⁸Glu²²-GLP-1(7-36)amide, Val⁸Glu²²-GLP-1(7-37), Val⁸Lys²²-GLP-1(7-36)-amide, Val⁸Lys²²-GLP-1(7-37), Val⁸Arg²²-GLP-1(7-36)-amide, Val⁸Arg²²-GLP-1(7-37), Val⁸His²²-GLP-1(7-36)-amide, Val⁸His²²-GLP-1(7-37), analogues thereof and derivatives of said agonists or analogues thereof.

39. The method according to claim 35, wherein the GLP-1 agonist is a stable GLP-1 analogue or derivative.

40. The method according to claim 30, wherein the GLP-1 agonist is exendin-4, an exendin-4 analogue or a derivative of said exendin-4 or exendin-4 analogue.

41. The method according to claim 40, wherein the GLP-1 agonist is a stable exendin-4 analogue or derivative.

42. The method according to claim 30, wherein the GLP-1 agonist or a pharmaceutically acceptable salt thereof is administered as a parenteral composition.

43. The method according to claim 42, wherein the composition comprises a buffer, an isotonicity agent and a preservative.

44. The method according to claim 42, wherein the composition is administered intravenously or subcutaneously.

45. The method according to claim 42, wherein the composition is administered by injection.

46. The method according to claim 42, wherein the composition is administered by infusion.

47. The method according to claim 30, wherein the dosage of GLP-1 agonist is from about 0.5 μg/kg/day to about 20 μg/kg/day.

48. The method according to claim 30, wherein the dosage of GLP-1 agonist is from about 0.1 μg/kg/day to about 2 μg/kg/day.

49. The method according to claim 30, wherein the GLP-1 agonist or pharmaceutically acceptable salt thereof is administered to the patient for more than 1 week.

50. The method according to claim 30, said method further comprising administering to said patient one or more pharmaceutical agents.

51. The method according to claim 50, wherein said agent is selected from the group consisting of anti-diabetic agents, anti-obesity agents, lipid modulating agents, anti-hypertensive agents and antiosteoporosis agents.

52. The method according to claim 51, wherein the anti-hypertensive agent is an angiotensin converting enzyme inhibitor.

53. The method according to claim 52, wherein the angiotensin converting enzyme inhibitor is selected from the group consisting of captopril, enalapril, fosinoprol, lisnoprol, quinapril, ramipril and spirapril.

54. The method according to claim 51, wherein the anti-hypertensive agent is an angiotensin II receptor antagonist.

55. The method according to claim 51, wherein the anti-hypertensive agent is a non-subtype-selective β-adrenergic antagonist.

56. The method according to claim 55, wherein the non-subtype-selective β-adrenergic antagonist is selected from the group consisting of propranolol, nadolol, timolol and pindolol.

57. The method according to claim 51, wherein the the antihypertensive agent is a selective β₁-adrenergic antagonist.

58. The method according to claim 57, wherein the selective β₁-adrenergic antagonist is selected from the group consisting of metoprolol, atenolol, esmolol and acebutolol.

59. The method according to claim 30, wherein the GLP-1 agonist or pharmaceutically acceptable salt thereof is administered to the patient for more than 4 weeks.

60. The method according to claim 30, wherein the GLP-1 agonist or pharmaceutically acceptable salt thereof is administered to the patient for more than 3 months.

61. The method according to claim 30, wherein the GLP-1 agonist or pharmaceutically acceptable salt thereof is administered to the patient for more than 6 months.

62. The method according to claim 32, wherein the patient is a diabetic patient.

63. The method according to claim 32, wherein the patient is a non-diabetic patient.

64. The method according to claim 32, wherein said GLP-1 agonist is selected from the group consisting of GLP-1(7-36)-amide, GLP-1(7-37), a GLP-1(7-36)-amide analogue, a GLP-1(7-37) analogue, or a derivative of any of these.

65. The method according to claim 64, wherein the GLP-1 agonist is a derivative of GLP-1(7-36)-amide, GLP-1(7-37), a GLP-1(7-36)-amide analogue or a GLP-1(7-37) analogue, which comprises a lipophilic substituent.

66. The method according to claim 65, wherein the GLP-1 agonist is Arg³⁴, Lys²⁶(N^{ε}-(γ-Glu(N^{α}-hexadecanoyl)))-GLP-1(7-37).

67. The method according to claim 64, wherein the GLP-1 agonist is selected from the group consisting of Gly⁸-GLP-1(7-36)-amide, Gly⁸-GLP-1(7-37), Val⁸-GLP-1(7-36)-amide, Val⁸GLP-1(7-37), Val⁸Asp²²-GLP-1(7-36)-amide, Val⁸Asp²²-GLP-1(7-37), Val⁸Glu²²-GLP-1(7-36)amide, Val⁸Glu²²-GLP-1(7-37), Val⁸Lys²²-GLP-1(7-36)-amide, Val⁸Lys²²-GLP-1(7-37), Val⁸Arg²²-GLP-1(7-36)-amide, Val⁸Arg²²-GLP-1(7-37), Val⁸His²²-GLP-1(7-36)-amide, Val⁸His²²-GLP-1(7-37), analogues thereof and derivatives of said agonists or analogues thereof.

68. The method according to claim 64, wherein the GLP-1 agonist is a stable GLP-1 analogue or derivative.

69. The method according to claim 32, wherein the GLP-1 agonist is exendin-4, an exendin-4 analogue or a derivative of said exendin-4 or exendin-4 analogue.

70. The method according to claim 69, wherein the GLP-1 agonist is a stable exendin-4 analogue or derivative.

71. The method according to claim 32, wherein the GLP-1 agonist or a pharmaceutically acceptable salt thereof is administered as a parenteral composition.

72. The method according to claim 71, wherein the composition comprises a buffer, an isotonicity agent and a preservative.

73. The method according to claim 71, wherein the composition is administered intravenously or subcutaneously.

74. The method according to claim 71, wherein the composition is administered by injection.

75. The method according to claim 71, wherein the composition is administered by infusion.

76. The method according to claim 32, wherein the dosage of GLP-1 agonist is from about 0.5 μg/kg/day to about 20 μg/kg/day.

77. The method according to claim 32, wherein the dosage of GLP-1 agonist is from about 0.1 μg/kg/day to about 2 μg/kg/day.

78. The method according to claim 32, wherein the GLP-1 agonist or pharmaceutically acceptable salt thereof is administered to the patient for more than 1 week.

79. The method according to claim 32, said method further comprising administering to said patient one or more pharmaceutical agents.

80. The method according to claim 79, wherein said agent is selected from the group consisting of anti-diabetic agents, anti-obesity agents, lipid modulating agents, anti-hypertensive agents and antiosteoporosis agents.

81. The method according to claim 80, wherein the anti-hypertensive agent is an angiotensin converting enzyme inhibitor.

82. The method according to claim 81, wherein the angiotensin converting enzyme inhibitor is selected from the group consisting of captopril, enalapril, fosinoprol, lisnoprol, quinapril, ramipril and spirapril.

83. The method according to claim 80, wherein the anti-hypertensive agent is an angiotensin II receptor antagonist.

84. The method according to claim 80, wherein the anti-hypertensive agent is a non-subtype-selective β-adrenergic antagonist.

85. The method according to claim 84, wherein the non-subtype-selective β-adrenergic antagonist is selected from the group consisting of propranolol, nadolol, timolol and pindolol.

86. The method according to claim 80, wherein the the antihypertensive agent is a selective β-adrenergic antagonist.

87. The method according to claim 86, wherein the selective β₁-adrenergic antagonist is selected from the group consisting of metoprolol, atenolol, esmolol and acebutolol.

88. The method according to claim 32, wherein the GLP-1 agonist or pharmaceutically acceptable salt thereof is administered-to the patient for more than 4 weeks.

89. The method according to claim 32, wherein the GLP-1 agonist or pharmaceutically acceptable salt thereof is administered to the patient for more than 3 months.

90. The method according to claim 32, wherein the GLP-1 agonist or pharmaceutically acceptable salt thereof is administered to the patient for more than 6 months.
